(19) United States
(12) Patent Application Publication
(10) Pub. No.: US 2020/0367776 A1
(43) Pub. Date: Nov. 26, 2020

(54) BIOSIGNAL MEASUREMENT DEVICE, ELECTROENCEPHALOGRAPH, AND CONTROL METHOD

(71) Applicant: PANASONIC INTELLECTUAL PROPERTY MANAGEMENT CO., LTD., Osaka (JP)

(72) Inventor: Akinori MATSUMOTO, Osaka (JP)

(21) Appl. No.: 16/959,788

(22) PCT Filed: Jan. 23, 2019
(86) PCT No.: PCT/JP2019/002007

§ 371 Date: Jul. 2, 2020

(30) Foreign Application Priority Data
Feb. 22, 2018 (JP) 2018-030084

(51) Int. Cl. A61B 5/04; A61B 5/0478; A61B 5/00

(52) U.S. Cl. CPC A61B 5/04004; A61B 5/0478; A61B 2562/166; A61B 5/7228; A61B 5/6801; A61B 5/7225

(57) ABSTRACT

A biosignal measurement device includes: a first chopper amplifier circuit that receives a biosignal detected by a measurement electrode contacting a living body, and is chopper-controlled based on a first control signal; and a controller that selectively performs one of a biosignal measurement mode operation for outputting the first control signal having a first frequency to the first chopper amplifier circuit and a test mode operation for outputting the first control signal having a different frequency from the first frequency to the first chopper amplifier circuit.

### TECHNICAL FIELD

The present invention relates to a biosignal measurement device, an electroencephalograph, a control method, and so forth. In particular, the invention relates to technology that is used for an operation test of the biosignal measurement device.

### BACKGROUND ART

A biosignal measurement device is known that measures the brainwaves or the heart rate, etc. of a test subject as biosignals. An example of such biosignal measurement device is described in Patent Literature 1 which discloses a sleep gauge capable of simple observation of a sleep state by frequency analysis of brainwaves.

### CITATION LIST

### Patent Literature

PTL 1: Japanese Unexamined Patent Application Publication No. 2007-105383

### SUMMARY OF THE INVENTION

### Technical Problem

In general, to conduct an operation test, the biosignal measurement device as described above requires a circuit that generates a test signal for the operation test.

The present invention provides a biosignal measurement device, an electroencephalograph, a control method, and a program that enable an operation test that uses a test signal, while preventing an increase in the circuit scale.

### Solutions to Problem

The biosignal measurement device according to one aspect of the present invention includes: a first chopper amplifier circuit that receives a biosignal detected by a first electrode contacting a living body, and is chopper-controlled based on a first control signal; and a controller that selectively performs one of a biosignal measurement mode operation for outputting the first control signal having a first frequency to the first chopper amplifier circuit and a test mode operation for outputting the first control signal having a different frequency from the first frequency to the first chopper amplifier circuit.

The electroencephalograph according to another aspect of the present invention includes the above biosignal measurement device, and an attachment portion via which the first electrode contacts the head portion of the living body.

The control method according to still another aspect of the present invention is a control method of controlling a biosignal measurement device that includes a first chopper amplifier circuit that receives a biosignal detected by a first electrode contacting a living body, and is chopper-controlled based on a first control signal. Such control method includes: selectively performing one of a biosignal measurement mode operation for outputting the first control signal having a first frequency to the first chopper amplifier circuit; and a test mode operation for outputting the first control signal having a different frequency from the first frequency to the first chopper amplifier circuit.

The program according to still another aspect of the present invention is a program that causes a computer to execute the above control method.

### Advantageous Effects of Invention

The biosignal measurement device, the electroencephalograph, the control method, and the program according to the present invention enable an operation test that uses a test signal, while preventing an increase in the circuit scale.

### BRIEF DESCRIPTION OF DRAWINGS

FIG. 1 is an external view of the configuration of a biosignal measurement system according to the embodiment.

FIG. 2A is a diagram showing an exemplary shape and an exemplary schematic structure of a headphone-type headset.

FIG. 2B is a diagram showing an exemplary shape and an exemplary schematic structure of a headband-type headset.

FIG. 3A is a diagram showing a first exemplary shape of the contact surface of an electrode that contacts the skin of a test subject.

FIG. 3B is a diagram showing a second exemplary shape of the contact surface of an electrode that contacts the skin of the test subject.

FIG. 3C is a diagram showing a third exemplary shape of the contact surface of an electrode that contacts the skin of the test subject.

FIG. 3D is a diagram showing a fourth exemplary shape of the contact surface of electrodes that contact the skin of the test subject.

FIG. 3E is a diagram showing a fifth exemplary shape of the contact surface of an electrode that contacts the skin of the test subject.

FIG. 4 is a block diagram showing an overall configuration of the biosignal measurement system according to the embodiment.

FIG. 5 is a functional block diagram showing detailed structures of the headset and an information processing device.

FIG. 6 is a block diagram showing the hardware structure of the headset.

FIG. 7 is a block diagram showing the hardware structure of the information processing device.

FIG. 8 is a flowchart showing the flow of basic processes performed in the biosignal measurement system according to the embodiment.

FIG. 9 is a circuit block diagram showing a detailed structure of the biosignal measurement device according to the embodiment.

FIG. 10 is a schematic diagram showing the noise levels of signals outputted from a first chopper amplifier circuit and a second chopper amplifier circuit.

FIG. 11 is a flowchart of a first exemplary operation for mode switching.

FIG. 12 is a diagram showing a status of the biosignal measurement device in the first exemplary operation.

FIG. 13 is a flowchart of a second exemplary operation for mode switching.

FIG. 14 is a diagram showing a status of the biosignal measurement device in the second exemplary operation.

FIG. 15 is a diagram showing a detailed structure of a biosignal processing unit.

FIG. 16 is a flowchart of an exemplary display operation performed in the biosignal measurement system according to the embodiment.

FIG. 17 is a diagram showing an exemplary display on a presentation unit in a biosignal measurement mode.

FIG. 18 is a diagram showing an exemplary display on the presentation unit in a test mode.

FIG. 19A is a first schematic diagram showing an external view of an active electrode.

FIG. 19B is a second schematic diagram showing an external view of the active electrode.

### DESCRIPTION OF EXEMPLARY EMBODIMENT

The following specifically describes the embodiment with reference to the drawings. Note that the following embodiment shows a comprehensive or specific illustration. The numerical values, shapes, materials, structural components, the arrangement and connection of the structural components, steps, the processing order of the steps, etc. shown in the following embodiment are mere examples, and thus are not intended to limit the present invention. Of the structural components described in the following embodiment, structural components not recited in any one of the independent claims that indicate the broadest concepts will be described as optional structural components.

Note that the drawings are schematic diagrams, and thus they are not necessarily precise illustrations. Also, substantially the same structural components are assigned the same reference marks throughout the drawings, and their repetitive descriptions may be omitted or simplified.

### Embodiment

### [Overview of Biosignal Measurement System]

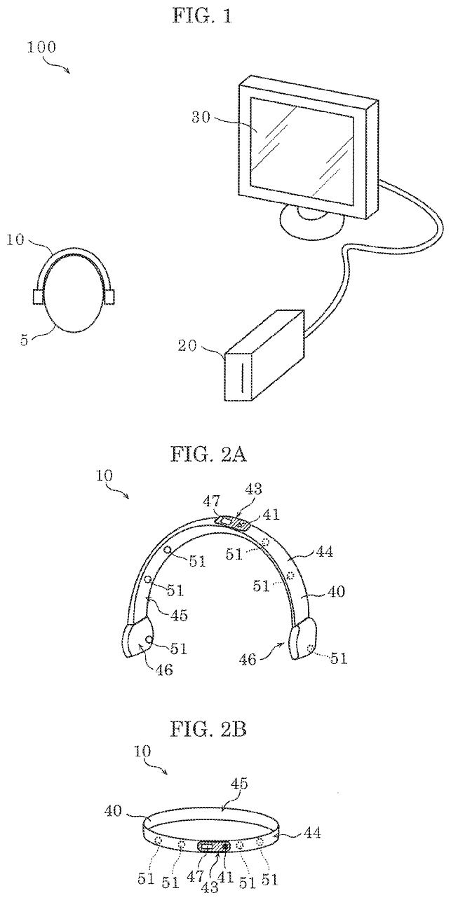

FIG. 1 is an external view of the configuration of biosignal measurement system 100 according to the embodiment. FIG. 1 also illustrates test subject 5 to be a target of measurement.

Biosignal measurement system 100, which is a system for measuring biosignals of test subject 5, includes headset 10, information processing device 20, and presentation unit 30. Headset 10, information processing device 20, and presentation unit 30 are interconnected via wired communication or wireless communication to transmit information therebetween.

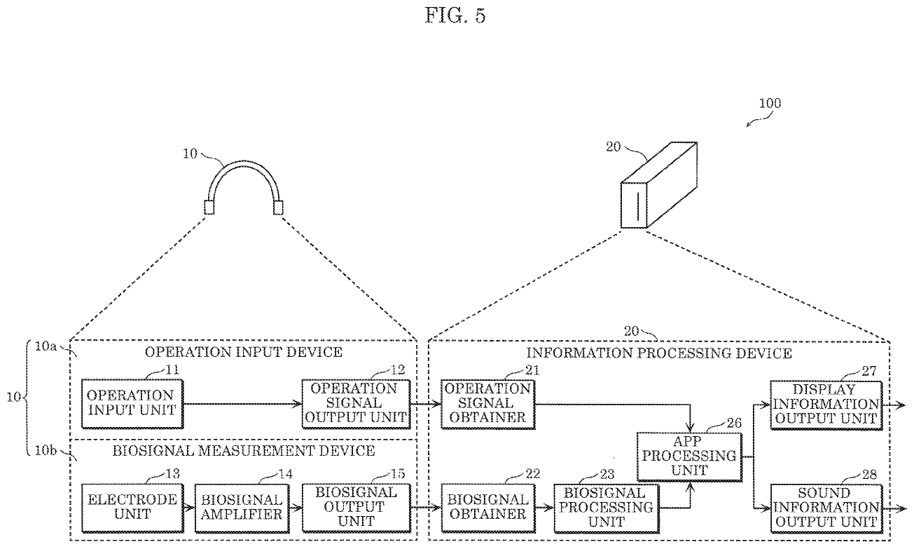

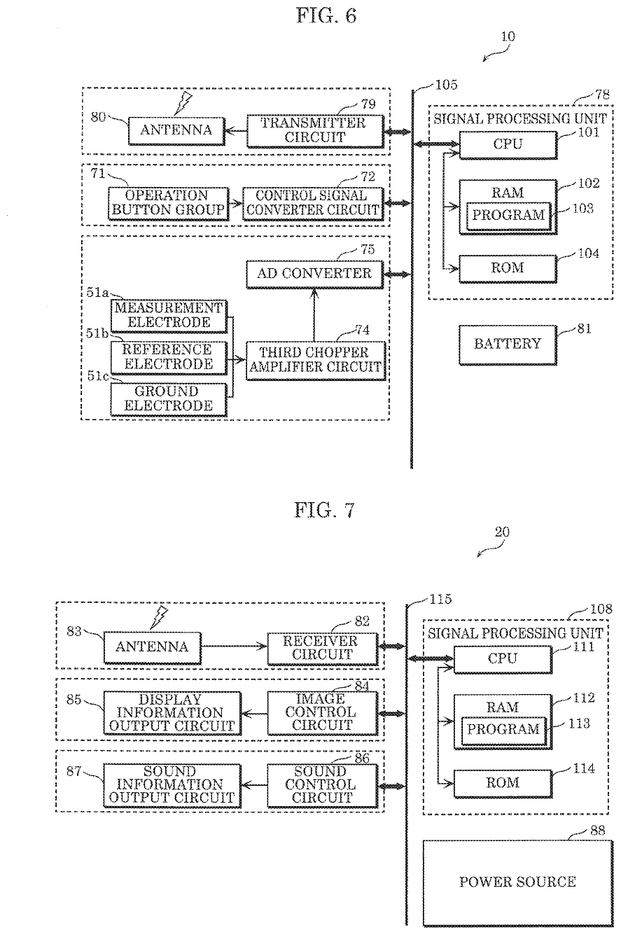

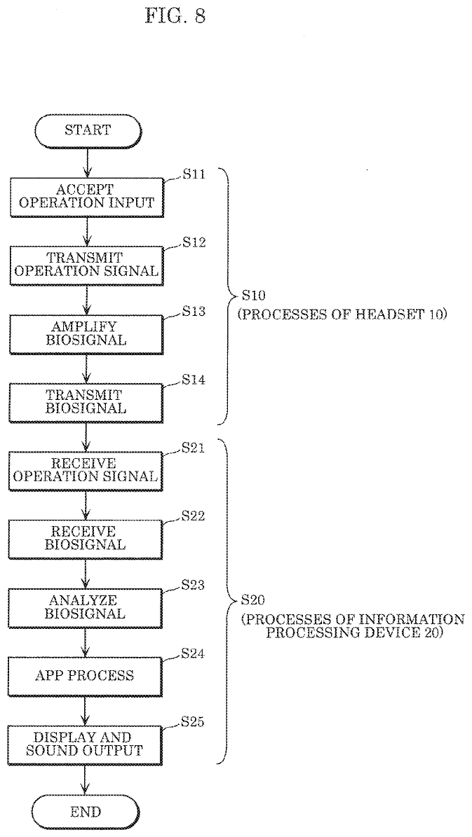

Headset 10, which is an exemplary device that detects biosignals, has the structure of the electroencephalograph to be described later. A plurality of electrodes 51 (refer to FIG. 2A and FIG. 2B) are attached to the head portion of test subject 5. Amongst a plurality of electrodes 51 are measurement electrode 51a that measures a biosignal potential and reference electrode 51b that measures the reference potential used to calculate the difference from the biosignal potential measured by the measurement electrode. Headset 10 also includes operation input device 10a (refer to FIG. 5) used by test subject 5 to input operation information to biosignal measurement system 100. An operation required to achieve a desired process is inputted to operation input device 10a. Note that the biosignal measurement device that is included in biosignal measurement system 100 is not limited to an electroencephalograph, and thus may be an electrocardiograph that detects electrocardiogram (ECG) signals from electrodes placed on the body, hands, limbs, and so forth.

Information processing device 20 accepts an operation input from headset 10 and performs predetermined processes. Information processing device 20 may be, for example, a computer. “Predetermined processes” here is a collective term of application processes that are executed in a home-use computer, such as a game, health management, and learning.

Presentation unit 30 is an output device that presents the result of the processes performed by information processing device 20. To “present” here refers to both displaying video on a display and/or outputting sound from a speaker. Stated differently, presentation unit 30 is a display and/or a speaker for displaying image information or outputting sound information.

### [Structure of Headset]

FIG. 2A and FIG. 2B are diagrams showing exemplary shapes and exemplary schematic structures of headset 10. FIG. 2A illustrates a headphone-type headset and FIG. 2B illustrates a headband-type headset. Test subject 6 attaches headset 10 as illustrated in FIG. 2A/FIG. 2B to the head portion.

Headset 10 illustrated in FIG. 2A has an arched headphone shape that fits along the head portion of test subject 5. As illustrated in FIG. 2A, headset includes a plurality of electrodes 51, attachment portion 40, and ear pads 46.

Attachment portion 40 is an arched member which is attached to the head portion of test subject 5 and on which electrodes 51 are provided. Attachment portion 40 includes operation surface 43, external surface 44, and attachment surface 45. External surface 44 is a surface that is on the opposing side of the head portion of test subject 5 who is wearing headset 10. Attachment surface 45 is a surface that is on the side of the head portion of test subject 5 who is wearing headset 10. Operation surface 43 includes operation button 41 and display 47. Electrodes 51 are provided on attachment surface of headset 10 and at ends of ear pads 46 that are on the same surface as that of attachment surface 45 of headset 10.

Before wearing headset 10, test subject 5 operates operation button 41 arranged on operation surface 43 to activate headset 10, and then attaches headset 10 to the head portion. Headset 10 is attached to the head portion of test subject 5 in a manner, for example, that left-hand ear pad 46 on the drawing sheet of FIG. 2A is located on the right ear of test subject 5 and right-hand ear pad 46 on the drawing sheet of FIG. 2A is located on the left ear of test subject 5. Ear pads 46 are placed in a manner that they cover the both ears of test subject 5. Electrodes 51 provided on attachment surface 45 are placed on the skin (i.e., head skin) of test subject 5. Electrodes 51 that are provided on ends of ear pads 46 are placed behind the ears of test subject 5. Electrode 51 that is provided on an end of left-hand ear pad 46 on the drawing sheet of FIG. 2A may serve as a ground electrode to be described later, electrode 51 that is provided on an end of right-hand ear pad 46 on the drawing sheet of FIG. 2A as a reference electrode to be described later, and the other electrodes 51 as measurement electrodes. The arrangement of the ground electrode and the reference electrode is not limited to the above positions, and thus electrode 51 that is provided on an end of right-hand ear pad 46 on the drawing sheet of FIG. 2A may serve as a ground electrode and electrode 51 that is provided on an end of left-hand ear pad 46 on the drawing sheet of FIG. 2A as a reference electrode.

Operation surface 43 displays on display 47 an operation status, the result of application processes, and so forth.

Headset 10 illustrated in FIG. 2B has a band shape that is wrapped around the head portion of test subject 5. Such headband-type headset 10 includes a plurality of electrodes 51 and attachment portion 40. Attachment portion 40 is a ring-shaped member which is attached to the head portion of test subject 5 and on which a plurality of electrodes 51 are provided. Attachment portion 40 includes operation surface 43, external surface 44, and attachment surface 45. The structures of electrodes 51 and operation surface 43 are similar to those of the headphone-type headset 10 illustrated in FIG. 2A.

Before wearing headset 10, test subject 5 operates operation button 41 arranged on operation surface 43 to activate headset 10, and then attaches headset 10 in a manner that the half of external surface 44 (operation surface 43 side) of the headband-type headset 10 comes on the forehead of test subject 5. Electrodes 51 are arranged on attachment surface 45 to contact the forehead of test subject 5. Note that among a plurality of electrodes 51, electrode 51 corresponding to the ground electrode and electrode 51 corresponding to the reference electrode may be placed behind the ears via lead wires (not illustrated) extended from attachment surface 45. Operation surface 43 further includes display 47 that displays an operation status and the result of application process. Note that the ground electrode does not refer to an electrode having a ground potential but an electrode having a potential that serves as the reference potential for test subject 5.

### [Shape of Electrode]

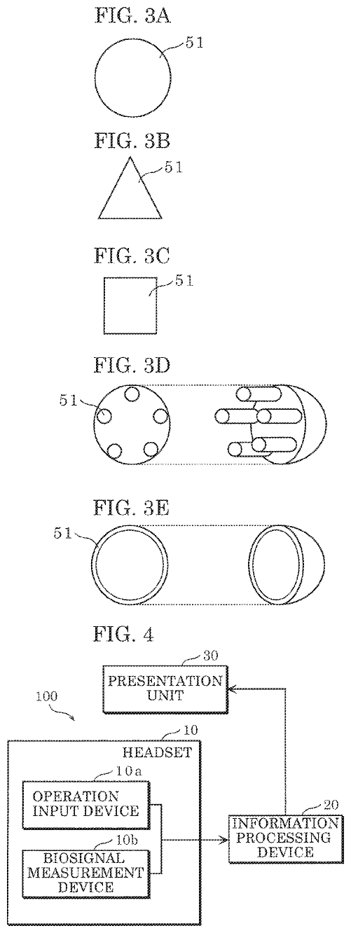

FIG. 3A through FIG. 3E are diagrams showing exemplary shapes of the contact surface of electrode 51 that contacts the skin of test subject 5. The material of electrode 51 includes a conducting substance. Exemplary materials of electrode 51 include gold and silver. The material of electrode 51 may be silver-silver chloride (Ag/AgCl). This is because silver-silver chloride is less likely to be polarized and generates a stable polarization voltage when in contact with a living body.

The contact surface of electrode 51 may have a circular shape illustrated in FIG. 3A (having an exemplary diameter of 10 mm) that is similar to a medical electrode or may have other different shapes depending on its usage. For example, the contact surface of electrode 51 may have a triangle shape as illustrated in FIG. 3B or may have a boxy or square shape as illustrated in FIG. 3C.

As illustrated in FIG. 3D, electrodes 51 arranged on attachment surface 45 of the headphone-type headset 10 may be formed as a plurality of cylinders (five cylinders in the drawing). Electrodes 51 having this structure can be combed through the hair of test subject 5 to come in contact with the skin of test subject 5. That contact surface of each cylinder which contacts the skin may have a circular shape as illustrated in FIG. 3D, or another shape such as an oval shape. Also, the shape of electrode 51 is not limited to a cylindric shape, and thus may be a prism. The number of cylinders or prisms may be, but is not limited to, five as illustrated in FIG. 3D, and thus any other numbers of cylinders or prisms may be used as appropriate. Also, that contact surface of the tip of each cylinder illustrated in FIG. 3D which contacts the skin may have a chamfered (i.e., rounded) shape. This structure increases the area in which each cylinder contacts the skin.

As illustrated in FIG. 3E, the surface of electrode 51 that contacts the skin of test subject 5 may also have a concentric circular shape. Electrodes 51 having such shape are used, for example, for ear pads 46 of the headphone-type headset 10 illustrated in FIG. 2A or the headband-type headset 10 illustrated in FIG. 2B to come in contact with the forehead, behind the ears, or other hairless portions. Electrode 51 having the shape as illustrated in FIG. 3E reduces the pressure on the skin compared to electrodes 51 having the shape as illustrated in FIG. 3D, and thus alleviates the load on test subject 5.

### [Configuration of Biosignal Measurement System]

FIG. 4 is a block diagram showing an overall configuration of biosignal measurement system 100. As described above, biosignal measurement system 100 includes headset 10, information processing device 20, and presentation unit 30. Headset 10 includes operation input device 10a and biosignal measurement device 10b.

In headset 10, operation input device 10a accepts an operation input from test subject 5, and biosignal measurement device 10b measures biosignals of test subject 5 at the time of performing the operation. Each biosignal measured by headset 10 is transmitted to information processing device 20.

Information processing device 20 performs predetermined processes upon receipt of an input from operation input device 10a or biosignal measurement device 10b, and outputs the result of the processes to presentation unit 30. Headset 10 and information processing device 20 are connected via wireless communication or wired communication.

FIG. 5 is a functional block diagram showing detailed structures of headset 10 and information processing device 20. The following description explains an example case where headset 10 and information processing device are connected via wireless communication.

Operation input device 10a includes operation input unit 11 and operation signal output unit 12.

Operation input unit 11 is an input device that obtains operation input information inputted from operation button 41 (refer to FIG. 2A and FIG. 2B) and interprets the details of the operation. Operation signal output unit 12 is a transmitter that transmits the operation input information obtained by operation input unit 11 to information processing device 20. Operation signal output unit 12 transmits the operation input information obtained by operation input unit 11 to information processing device 20.

Biosignal measurement device 10b includes electrode unit 13, biosignal amplifier 14, and biosignal output unit 15.

Electrode unit 13 includes a plurality of electrodes 51. A plurality of electrodes 51 include the measurement electrodes and the reference electrode as described above. A plurality of electrodes 51 are placed, for example, on positions that contact the skin of test subject 5.

Biosignal amplifier 14 is an amplifier that amplifies a biosignal which is equivalent to the potential difference between a plurality of electrodes 51. More specifically, biosignal amplifier 14 measures the potential difference between measurement electrode 51a (refer to FIG. 6) placed on the skin of test subject 5 and reference electrode 51b placed behind an ear of test subject 5 (refer to FIG. 6) among a plurality of electrodes 51, and amplifies such measured potential difference. The amplified potential difference is converted into a digital signal, for example, by an AD converter (not illustrated) provided in biosignal amplifier 14. Biosignal output unit 15 is a transmitter that transmits the potential difference amplified by biosignal amplifier 14 to information processing device 20. Biosignal output unit 15 transmits to information processing device 20 the potential difference of the biosignal converted into a digital value by biosignal amplifier 14.

Note that biosignal amplifier 14 is not required to amplify a biosignal when a biosignal with a potential of a predetermined magnitude or greater is measurable. In this case, biosignal amplifier 14 is simply required to measure the potentials of a plurality of electrodes 51.

Information processing device 20 includes operation signal obtainer 21, biosignal obtainer 22, biosignal processing unit 23, application processing unit (app processing unit) 26, display information output unit 27, and sound information output unit 28.

In information processing device 20, operation signal obtainer 21 receives the operation input information and biosignal obtainer 22 receives each biosignal, thereby receiving information from headset 10.

In many cases, a biosignal as is, i.e., a primary signal which is simply recorded, cannot be used as information. For this reason, biosignal processing unit 23 performs a process of extracting meaningful information from the primary signal. In the case of brainwave measurement, for example, biosignal processing unit 23 extracts a signal having a specific frequency (e.g., 10 Hz) and calculates the power spectral density of the signal at such frequency. Note that biosignal processing unit 23 may be placed not in information processing device 20 but in headset 10. Stated differently, in the present embodiment, headset 10 and biosignal processing unit 23 may form an electronic device.

Application processing unit 26 performs main application processes (app processes) of information processing device 20. The execution of application processes are achieved by performing predetermined processes upon receipt of a signal input from headset 10. Predetermined processes refer to, for example, the proceeding of a game in a game application, data recording/management/display in a health management application, the presentation of test questions/scoring/result display in a learning application, and so forth.

Application processing unit 26 outputs the result of the processes application processing unit 26 has performed to display information output unit 27 and sound information output unit 28. To provide to test subject 5 the feedback about the result of the processes performed by application processing unit 26, display information output unit 27 and sound information output unit 28 output a visual signal/auditory signal to presentation unit 30.

Presentation unit 30 presents signals outputted from display information output unit 27 and sound information output unit 28 (i.e., display and/or sound output). Through this, the signals are presented to test subject 5. Presentation unit 30 is, for example, a television, a display, or a speaker.

### [Hardware Structure of Headset]

FIG. 6 is a block diagram showing the hardware structure of headset 10. Headset 10 includes operation button group 71, control signal converter circuit 72, measurement electrode 51a, reference electrode 51b, ground electrode 51c, third chopper amplifier circuit 74, AD converter 75, transmitter circuit 79, signal processing unit 78, antenna 80, and battery 81.

Of these structural components, operation button group 71 and control signal converter circuit 72 correspond to operation input unit 11 illustrated in FIG. 5. Each button included in operation button group 71 corresponds to operation button 41. Measurement electrode 51a, reference electrode 51b, and ground electrode 51c correspond to electrodes 51 illustrated in FIG. 2A and FIG. 2B, and electrode unit 13 illustrated in FIG. 5. Third chopper amplifier circuit 74 and AD converter 75 are included in biosignal amplifier 14.

Signal processing unit 78 includes CPU 101, RAM 102, program 103, and ROM 104. Transmitter circuit 79 and antenna 80 function as biosignal output unit 15 and/or operation signal output unit 12 illustrated in FIG. 5. Transmitter circuit 79 and antenna 80 may also be referred to as “output unit” or “transmitter”.

Interconnected via bus 105, these structural components are capable of transmitting data between one another. Headset 10 operates, with battery 81 serving as the power source.

Control signal converter circuit 72 converts information on the pressing of each button of operation button group 71 into a control signal for controlling the operation of headset 10. The control signal is then transmitted to CPU 101 via bus 105.

Third chopper amplifier circuit 74 is connected to measurement electrode 51a, reference electrode 51b, and ground electrode 51c directly or via a buffer amplifier, etc. These electrodes are arranged on predetermined positions in headset 10. Third chopper amplifier circuit 74 amplifies the potential difference between measurement electrode 51a and reference electrode 51b, and AD converter 75 converts the amplified potential difference from an analog biosignal to a digital biosignal. The digital biosignal converted from the potential difference is then transmitted to CPU 101 via bus 105 as a processable, transmittable biosignal.

CPU 101 executes program 103 stored in RAM 102. Program 103 describes the order of signal processes performed in headset 10 shown in the flowchart of FIG. 8 to be described later. Headset 10 converts the operation signal and each biosignal into digital signals according to such program 103, and transmits the resulting digital signals from antenna 80 via transmitter circuit 79. Program 103 may also be stored in ROM 104.

Note that signal processing unit 78, control signal converter circuit 72, transmitter circuit 79, third chopper amplifier circuit 74, and AD converter 75 may be implemented as hardware such as a digital signal processor (DSP) that is achieved by embedding a computer program in a single semiconductor integrated circuit. Packaging of structural components in a single semiconductor integrated circuit achieves reduction in footprint and thus in power consumption.

Also, third chopper amplifier circuit 74 and AD converter 75 may be packaged in a single semiconductor integrated circuit, and signal processing unit 78, control signal converter circuit 72, and transmitter circuit 79 may be packaged in another semiconductor integrated circuit. These two semiconductor integrated circuits are then connected in a single package as a system in package (SiP), so that these structural components are implemented as hardware such as a DSP that is embedded with a computer program. Packaging of structural components in two semiconductor integrated circuits that are manufactured in different manufacture processes achieves cost reduction compared to packaging of structural components in a single semiconductor integrated circuit.

### [Hardware Structure of Information Processing Device]

FIG. 7 is a block diagram showing the hardware structure of information processing device 20. Information processing device 20 includes antenna 83, receiver circuit 82, signal processing unit 108, image control circuit 84, display information output circuit 85, sound control circuit 86, sound information output circuit 87, and power source 88. Of these structural components, antenna 83 and receiver circuit 82 correspond to biosignal obtainer 22 and/or operation signal obtainer 21 illustrated in FIG. 5. These structural components may also be referred to as “receivers”.

Signal processing unit 108 includes CPU 111, RAM 112, program 113, and ROM 114. Signal processing unit 108 corresponds to biosignal processing unit 23 and/or application processing unit 26 illustrated in FIG. 5. Image control circuit 84 and display information output circuit 85 correspond to display information output unit 27 illustrated in FIG. 5. Sound control circuit 86 and sound information output circuit 87 correspond to sound information output unit 28 illustrated in FIG. 5. Interconnected via bus 115, these structural components are capable of transmitting data between one another. Power source 88 supplies power to each circuit.

Receiver circuit 82 receives operation information and living body information from headset 10 via antenna 83. These items of information are then transmitted to CPU 111 via bus 115.

CPU 111 executes program 113 stored in RAM 112. Program 113 describes the order of signal processes performed in information processing device 20 shown in the flowchart of FIG. 8 to be described later. Information processing device 20 converts the operation signal and each biosignal according to program 113, preforms a process of executing a predetermined application, and generates signals used to provide feedback to test subject 5 in the form of image/sound. Program 113 may also be stored in ROM 114.

Display information output circuit 85 outputs an image feedback signal generated by signal processing unit 108 to presentation unit 30 via image control circuit 84. Similarly, sound information output circuit 87 outputs a sound feedback signal generated by signal processing unit 108 via sound control circuit 86.

Note that signal processing unit 108, receiver circuit 82, image control circuit 84, and sound control circuit 86 may be implemented as hardware such as a DSP in which a program is embedded in a single semiconductor integrated circuit. Packaging of structural components in a single semiconductor integrated circuit achieves reduction in power consumption.

### [Operation of Biosignal Measurement System]

The following describes the operation performed in biosignal measurement system 100 according to the present embodiment having the above configuration.

FIG. 8 is a flowchart showing the flow of basic processes performed in biosignal measurement system 100. Step S11 through step S14 indicate the processes performed in headset 10 (step S10), and step S21 through step S25 indicate the processes performed in information processing device 20 (step S20).

First, the processing step S10 performed in headset 10 will be described.

### <Step S11>

Operation input unit 11 accepts an operation input performed by test subject 5. More specifically, operation input unit 11 detects which operation button 41 is being pressed at the timing of accepting the input. Exemplary timing of accepting the input is the timing at which operation button 41 was pressed. Whether operation button 41 has been pressed is detected, for example, by detecting mechanical changes in the button position or changes in an electric signal when operation button 41 has been pressed. Operation input unit 11 also detects the type of the operation input which operation input unit 11 has accepted, on the basis of the type of operation button 41 having been pressed, and transmits the detected type to operation signal output unit 12.

### <Step S12>

Operation signal output unit 12 transmits to information processing device 20 an operation signal corresponding to the operation input accepted by operation input unit 11.

### <Step S13>

Biosignal amplifier 14 measures and amplifies a biosignal that is equivalent to the potential difference between a plurality of electrodes 51 in electrode unit 13. For example, biosignal amplifier 14 measures the potential difference between measurement electrode 51a placed on the right side of the head portion (the electrode location C4 described in International 10-20 system) and reference electrode 51b, among a plurality of electrodes 51 in electrode unit 13. Biosignal amplifier 14 also amplifies the measured biosignal. Biosignal amplifier 14 transmits the amplified biosignal to biosignal output unit 15.

### <Step S14>

Further, biosignal output unit 15 sends the transmitted biosignal to information processing device 20.

Note that “step S11 and step S12” and “step S13 and step S14” in the processing step S10 performed in headset 10 may each be performed in parallel, and thus not all the processes of step S11 through step S14 are required to be performed in the stated order.

Next, the processing step S20 performed in information processing device 20 will be described.

### <Step S21>

In information processing device 20, operation signal obtainer 21 receives the operation signal from operation signal output unit 12. Operation signal obtainer 21 transmits the received operation signal to application processing unit 26.

### <Step S22>

Biosignal obtainer 22 receives the biosignal from biosignal output unit 15. Biosignal obtainer 22 transmits the received biosignal to biosignal processing unit 23.

### <Step S23>

Biosignal processing unit 23 performs a process of analyzing the biosignal received by biosignal obtainer 22 to extract meaningful information. For example, biosignal processing unit 23 extracts predetermined frequency components from the biosignal. Predetermined frequency components are frequency components at 10 Hz, for example, in the case of brainwave measurement.

### <Step S24>

Upon receipt of the operation signal from operation signal obtainer 21 and the biosignal from biosignal processing unit 23, application processing unit 26 performs predetermined processes for executing the current app. Predetermined processes refer to, for example, the proceeding of a game in a game application, data recording/management/display in a health management application, the presentation of test questions/scoring/result display in a learning application, and so forth.

### <Step S25>

To provide to test subject 5 the feedback about the result of the processes performed by application processing unit 26, display information output unit 27 outputs video information to presentation unit 30, and sound information output unit 28 outputs sound information to presentation unit 30. Through this process, presentation unit 30 outputs the image and the sound corresponding to the result of the processes.

Note that “step S22 and step S23”, and “step S24” in the processing step S20 performed in information processing device 20 may each be performed in parallel. Also, application processing unit 26 is not required to perform the processes using both the operation signal from operation signal obtainer 21 and the biosignal from biosignal processing unit 23, and thus may perform the processes using only the biosignal. In this case, step S21 of receiving the operation signal can be omitted.

The flow of the above processes enables biosignal measurement system 100 to obtain the living body information such as brainwaves and electrocardiogram from test subject 5.

### [Detailed Structure of Biosignal Measurement Device]

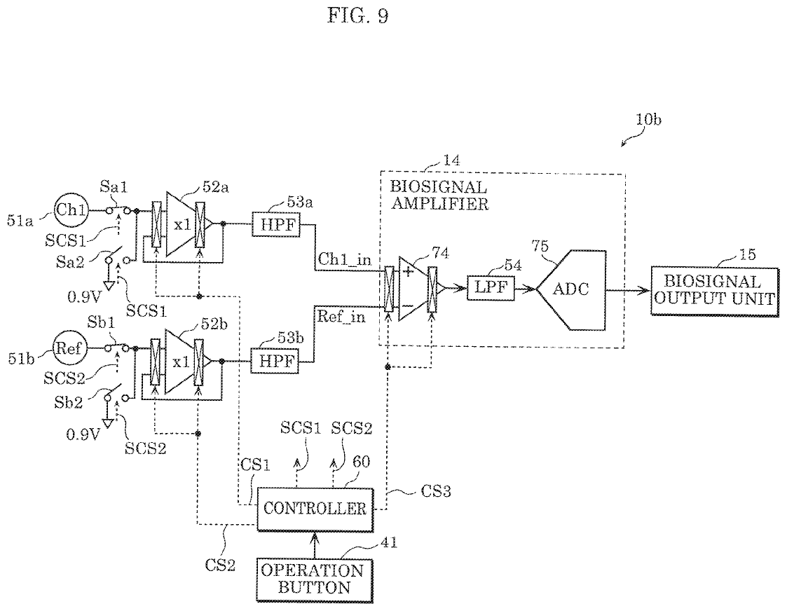

The following describes a detailed structure of biosignal measurement device 10b included in headset 10. FIG. 9 is a circuit block diagram showing a detailed structure of biosignal measurement device 10b included in headset 10. FIG. 9 shows the structure of hardware components that relate to biosignal measurement device 10b among the hardware components included in headset 10.

Biosignal measurement device 10b includes measurement electrode 51a, reference electrode 51b, switch element Sa1, switch element Sa2, switch element Sb1, switch element Sb2, first chopper amplifier circuit 52a, second chopper amplifier circuit 52b, first highpass filter 53a, second highpass filter 53b, biosignal amplifier 14, biosignal output unit 15, controller 60, and operation button 41.

Switch element Sa1 and switch element Sa2 switch between a biosignal detected by measurement electrode 51a and the reference voltage of the reference voltage source (e.g., 0.9 V) to be inputted to first chopper amplifier circuit 52a, in accordance with switch control signal SCS1 outputted from controller 60. Switch element Sa1 and switch element Sa2 are, for example, field effect transistors (FETs), but may be other switch elements.

First chopper amplifier circuit 52a is an amplifier that accepts an input of each biosignal detected by measurement electrode 51a that contacts the living body. Measurement electrode 51a is an example of the first electrode. First chopper amplifier circuit 52a functions as what is known as a buffer amplifier, and performs impedance conversion. First chopper amplifier circuit 52a performs no voltage amplification (its voltage amplification factor is one), but may perform voltage amplification. Note that the term “amplifier circuit” or “amplifier” in the present specification is not necessarily limited to an amplifier circuit or an amplifier having a voltage amplification factor greater than one, and thus also refers to an amplifier having a voltage amplification factor of one or smaller.

First chopper amplifier circuit 52a includes a modulation chopper circuit provided at the input and a demodulation chopper circuit provided at the output. These chopper circuits perform chopper control in accordance with the frequency of first control signal CS1 outputted from controller 60. Stated differently, the first chopper amplifier circuit is chopper-controlled on the basis of first control signal CS1. First control signal CS1 is basically a chopper clock that takes on either of the two values, high-level or low-level. Such chopper control reduces low-frequency noise.

Switch element Sb1 and switch element Sb2 switch between a biosignal detected by reference electrode 51b and the reference voltage of the reference voltage source (e.g., 0.9 V) to be inputted to second chopper amplifier circuit 52b, in accordance with switch control signal SCS2 outputted from controller 60. Switch element Sb1 and switch element Sb2 are, for example, FETs, but may be other switch elements.

Second chopper amplifier circuit 52b is an amplifier that accepts an input of each biosignal detected by reference electrode 51b that contacts the living body. Reference electrode 51b is an example of the second electrode. Second chopper amplifier circuit 52b functions as what is known as a buffer amplifier, and performs impedance conversion. Second chopper amplifier circuit 52b performs no voltage amplification (its voltage amplification factor is one), but may perform voltage amplification.

Second chopper amplifier circuit 52b includes a modulation chopper circuit provided at the input and a demodulation chopper circuit provided at the output. These chopper circuits perform chopper control in accordance with the frequency of second control signal CS2 outputted from controller 60. Stated differently, the second chopper amplifier circuit is chopper-controlled on the basis of second control signal CS2. Second control signal CS2 is basically a chopper clock that takes on either of the two values, high-level or low-level. Such chopper control reduces low-frequency noise.

First highpass filter 53a is a filter that rejects unnecessary low-frequency components included in the signal outputted from first chopper amplifier circuit 52a. First highpass filter 53a is, for example, a passive filter with a cutoff frequency of 0.5 Hz.

Second highpass filter 53b is a filter that rejects unnecessary low-frequency components included in the signal outputted from second chopper amplifier circuit 52b. Second highpass filter 53b is, for example, a passive filter with a cutoff frequency of 0.5 Hz.

Biosignal amplifier 14 includes third chopper amplifier circuit 74, lowpass filter 54, and AD converter 75.

Third chopper amplifier circuit 74 is an amplifier that amplifies the difference between output signal CH1_in from first highpass filter 53a and output signal Ref_in from second highpass filter 53b (i.e., potential difference). Stated differently, third chopper amplifier circuit 74 basically amplifies the difference between the signal outputted from first chopper amplifier circuit 52a and the signal outputted from second chopper amplifier circuit 52b. Through this process, third chopper amplifier circuit 74 outputs, as an amplified biosignal, a signal that is resulted from amplifying the potential of measurement electrode 51a that is based on the potential of reference electrode 51b. The voltage amplification factor of third chopper amplifier circuit 74 is, for example, 1200.

Third chopper amplifier circuit 74 includes a modulation chopper circuit provided at the input and a demodulation chopper circuit provided at the output. These chopper circuits performs chopper control in accordance with the frequency of third control signal CS3 outputted from controller 60. Stated differently, the third chopper amplifier circuit is chopper-controlled on the basis of third control signal CS3. Third control signal CS3 is basically a chopper clock that takes on either of the two values, high-level or low-level. Such chopper control reduces low-frequency noise.

Lowpass filter 54 is a filter that rejects unnecessary high-frequency components included in the signal outputted from third chopper amplifier circuit 74. Lowpass filter 54 is, for example, an active filter with a cutoff frequency of 100 Hz.

AD converter 75 is a converter that samples the signal outputted from lowpass filter 54 to convert it into, for example, a 12-bit digital signal at a sampling rate of 1 kHz.

Biosignal output unit 15 is a transmitter that transmits the potential difference amplified by biosignal amplifier 14 to information processing device as described above. Biosignal output unit 15 transmits to information processing device 20 the potential difference of the biosignal converted into the digital value by AD converter 75 of biosignal amplifier 14.

Controller 60 performs control of switching between the biosignal measurement mode operation and the test mode operation. Controller 60 is implemented, for example, as a microcomputer or a processor, but may be implemented as a dedicated circuit.

The biosignal measurement mode is, in other words, a normal mode in which third chopper amplifier circuit 74 amplifies the potential difference between measurement electrode 51a and reference electrode 51b and outputs the amplified potential difference. Stated differently, the biosignal measurement mode is a mode for measuring the brainwaves of test subject 5 as per normal.

In contrast, the test mode is a mode for conducting an operation test to check whether a signal is appropriately outputted from biosignal output unit (whether a signal can be monitored on presentation unit 30). In the test mode, headset 10 is not required to be attached to test subject 5.

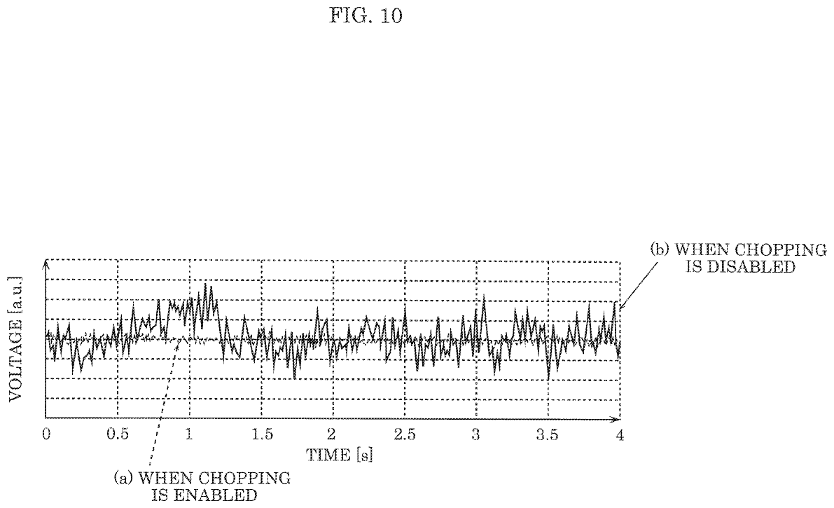

In biosignal measurement device 10b, first chopper amplifier circuit 52a and second chopper amplifier circuit 52b generate low-frequency noise as a result of stopping the respective chopper circuits of first chopper amplifier circuit 52a and second chopper amplifier circuit 52b. FIG. 10 is a schematic diagram showing the noise levels of signals outputted from first chopper amplifier circuit 52a and second chopper amplifier circuit 52b.

Noise is not required for the measurement of brainwaves of test subject in the biosignal measurement mode. For this reason, as shown in (a) in FIG. 10, the noise levels of the signals outputted from first chopper amplifier circuit 52a and second chopper amplifier circuit 52b are low when chopping is enabled. As such, in the biosignal measurement mode operation, controller 60 causes the respective chopper circuits of first chopper amplifier circuit 52a and second chopper amplifier circuit 52b to operate.

Meanwhile, as shown in (b) in FIG. 10, first chopper amplifier circuit 52a and second chopper amplifier circuit 52b generate low-frequency noise when chopping is disabled. In the test mode operation, controller 60 stops the respective chopper circuits of first chopper amplifier circuit 52a and second chopper amplifier circuit 52b to intentionally generate low-frequency noise. Such low-frequency noise is not completely canceled in third chopper amplifier circuit 74, and thus can be utilized as a test signal.

This structure enables a simple operation test to be conducted without adding a circuit for generating a test signal. It is particularly difficult to generate a test signal in the frequency band between 0.5 Hz and 8 Hz, inclusive, which is the frequency of brainwaves during sleep (e.g., δ waves and θ waves) and conduct an operation test, but biosignal measurement device 10b easily enables such an operation test.

### [First Exemplary Operation for Mode Switching]

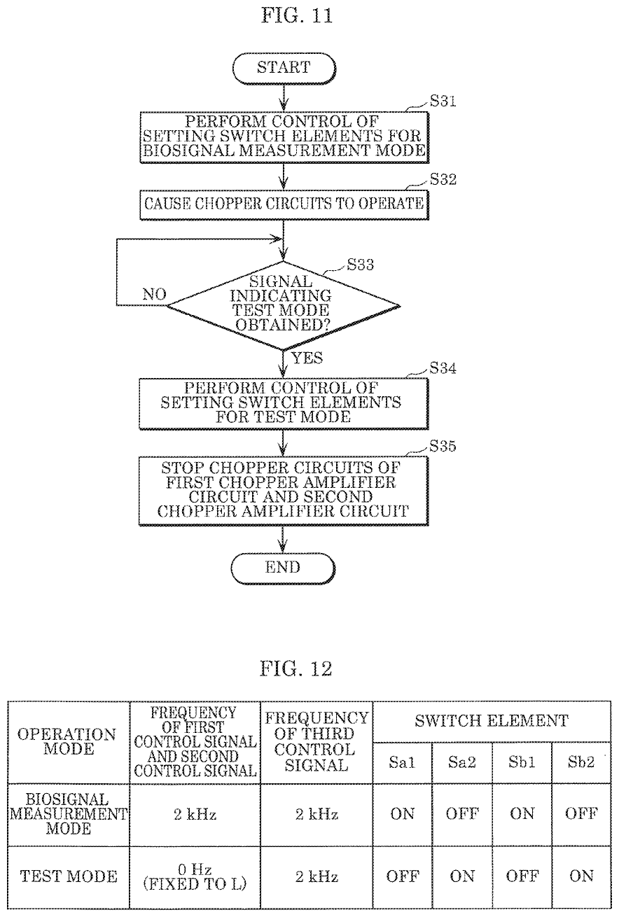

The following describes in detail a first exemplary operation for switching between the biosignal measurement mode and the test mode. FIG. 11 is a flowchart of the first exemplary operation for mode switching. FIG. 12 is a diagram showing a status of biosignal measurement device 10b in the first exemplary operation.

In the first exemplary operation, controller 60 first performs the operation for the biosignal measurement mode. Controller 60 performs control of setting switch element Sa1, switch element Sa2, switch element Sb1, and switch element Sb2 for the biosignal measurement mode (S31).

More specifically, controller 60 outputs switch control signal SCS1, thereby switching ON switch element Sa1 and switching OFF switch element Sa2. This process electrically connects measurement electrode 51a and first chopper amplifier circuit 52a, enabling a biosignal detected by measurement electrode 51a to be inputted to first chopper amplifier circuit 52a. Controller 60 also outputs switch control signal SCS2, thereby switching ON switch element Sb1 and switching OFF switch element Sb2. This process electrically connects reference electrode 51b and second chopper amplifier circuit 52b, enabling a biosignal detected by reference electrode 51b to be inputted to second chopper amplifier circuit 52b.

Controller 60 then causes the respective chopper circuits of first chopper amplifier circuit 52a, second chopper amplifier circuit 52b, and third chopper amplifier circuit 74 to operate (S32). More specifically, controller 60 outputs square waves having a frequency of 2 kHz as first control signal CS1, second control signal CS2, and third control signal CS3.

Subsequently controller 60 determines whether the signal indicating the test mode has been obtained (S33). This determination is continuously made until the signal indicating the test mode is obtained (NO in S33). The signal indicating the test mode is outputted from operation button 41 to controller 60 by, for example, an operation that designates the test mode being performed on operation button 41. The signal indicating the test mode may be outputted to controller 60 on the basis of an operation performed on a user interface (not illustrated) included in information processing device 20.

When determining that the signal indicating the test mode has been obtained (YES in S33), controller 60 performs the operation for the test mode. Controller 60 performs control of setting switch element Sa1, switch element Sa2, switch element Sb1, and switch element Sb2 for the test mode (S34).

More specifically, controller 60 outputs switch control signal SCS1, thereby switching OFF switch element Sa1 and switching ON switch element Sa2. This process electrically connects the reference voltage source and first chopper amplifier circuit 52a, enabling the reference voltage of 0.9 V to be inputted to first chopper amplifier circuit 52a. Controller 60 also outputs switch control signal SCS2, thereby switching OFF switch element Sb1 and switching ON switch element Sb2. This process electrically connects the reference voltage source and second chopper amplifier circuit 52b, enabling the reference voltage of 0.9 V to be inputted to second chopper amplifier circuit 52b.

Subsequently, controller 60 stops the respective chopper circuits of first chopper amplifier circuit 52a and second chopper amplifier circuit 52b (S35). More specifically, controller 60 outputs low-level signals having a frequency of 0 Hz (fixed to L) as first control signal CS1 and second control signal CS2. This enables the low-frequency noise generated in first chopper amplifier circuit 52a and second chopper amplifier circuit 52b to be utilized as a test signal. Such low-frequency noise is, for example, noise known as 1/f noise (or pink noise).

In step S35, high-level signals having a frequency of 0 Hz (fixed to H) may be outputted as first control signal CS1 and second control signal CS2. Also in step S35, one of first control signal CS1 and second control signal CS2 may be a low-level signal having a frequency of 0 Hz, and the other of first control signal CS1 and second control signal CS2 may be a high-level signal having a frequency of 0 Hz. Stated differently, complementary signals (high-level signal and low-level signal) may be outputted as first control signal CS1 and second control signal CS2.

In step S35, at least one of the chopper circuits of first chopper amplifier circuit 52a and the chopper circuits of second chopper amplifier circuit 52b is simply required to be stopped. Note that the chopper circuits may not be stopped in step S35. In the test mode, at least one of first chopper amplifier circuit 52a and second chopper amplifier circuit 52b is simply required to output a larger amount of low-frequency noise than in the normal mode.

For example, first chopper amplifier circuit 52a can generate a larger amount of low-frequency noise than in the normal mode by controller 60 outputting first control signal CS1 having a frequency higher than 2 kHz. Stated differently, in the test mode operation, controller 60 is simply required to output to first chopper amplifier circuit 52a first control signal CS1 having a different frequency from the frequency in the normal mode. This is applicable to second chopper amplifier circuit 52b.

### [Second Exemplary Operation for Mode Switching]

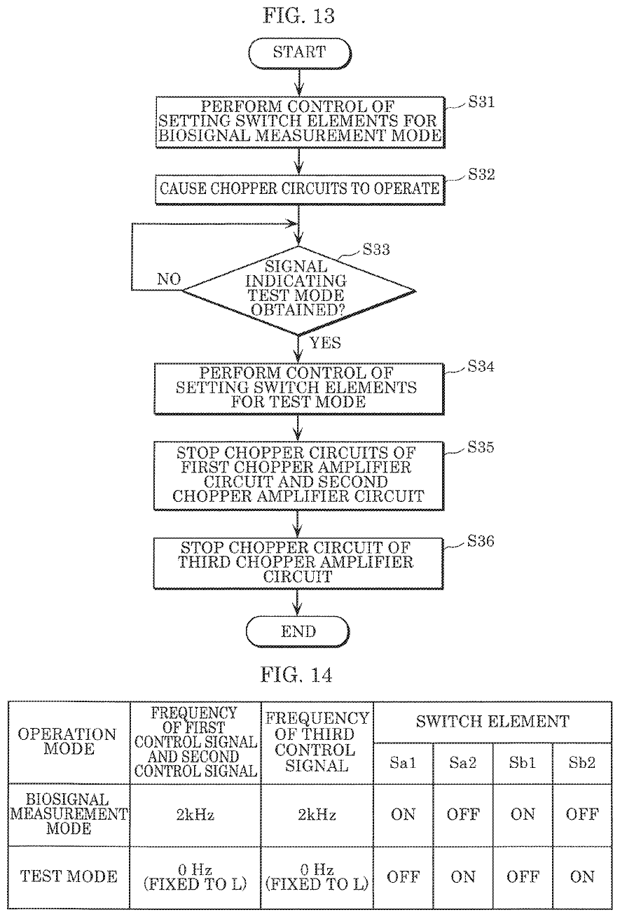

The following describes in detail a second exemplary operation for switching between the biosignal measurement mode and the test mode. FIG. 13 is a flowchart of the second exemplary operation for mode switching. FIG. 14 is a diagram showing a status of biosignal measurement device 10b in the second exemplary operation.

In the second exemplary operation, controller 60 stops not only the respective chopper circuits of first chopper amplifier circuit 52a and second chopper amplifier circuit 52b (S35), but also the chopper circuits of third chopper amplifier circuit 74 (S36). More specifically, controller 60 outputs a low-level signal having a frequency of 0 Hz (fixed to L) as third control signal CS3.

This enables the low-frequency noise generated in third chopper amplifier circuit 74 to be utilized as a test signal, in addition to the low-frequency noise generated in first chopper amplifier circuit 52a and second chopper amplifier circuit 52b. Such low-frequency noise is, for example, noise known as 1/f noise (or pink noise). In the second exemplary operation, the low-frequency noise generated in third chopper amplifier circuit 74 enables the obtainment of a test signal having a relatively great amplitude.

Note that the chopper circuits may not be stopped in step S36. In step S36, third chopper amplifier circuit 74 is simply required to output a larger amount of low-frequency noise than in the normal mode. For example, third chopper amplifier circuit 74 can generate a larger amount of low-frequency noise than in the normal mode by controller 60 outputting third control signal CS3 having a frequency higher than 2 kHz. Stated differently, in the test mode operation, controller 60 is simply required to output to third chopper amplifier circuit 74 third control signal CS3 having a different frequency from the frequency in the normal mode.

### [Exemplary Display Operation]

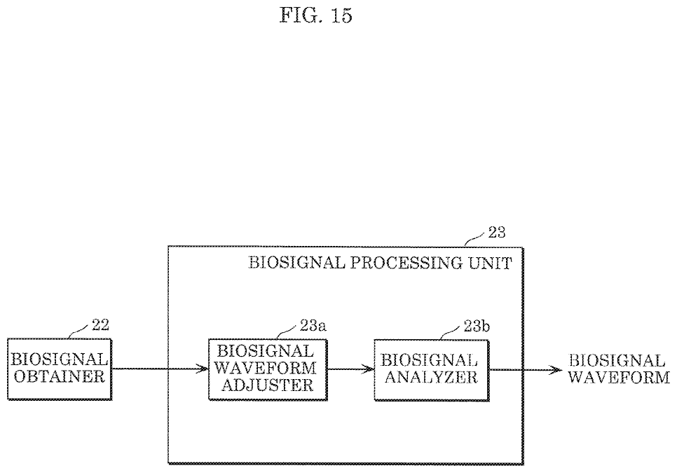

The following describes an exemplary operation performed in biosignal measurement system 100 for displaying the brainwave form of test subject 5. First, in relation to the display operation, a detailed structure of biosignal processing unit 23 of information processing device 20 will be described. FIG. is a diagram showing a detailed structure of biosignal processing unit 23. As illustrated in FIG. 15, biosignal processing unit 23 includes biosignal waveform adjuster 23a and biosignal analyzer 23b.

Biosignal waveform adjuster 23a performs waveform adjustment, such as amplitude adjustment, on each biosignal obtained by biosignal obtainer 22.

Biosignal analyzer 23b performs software-based filtering on the waveform-adjusted biosignal. Biosignal analyzer 23b functions, for example, as a highpass filter or a lowpass filter. The user can change the cutoff frequencies of the filter as appropriate. Biosignal analyzer 23b may include a notch filter that rejects only the frequency of humming noise (50 Hz or 60 Hz). Biosignal analyzer 23b performs signal processing by use of such filters and so forth to generate a biosignal waveform to be displayed by presentation unit 30 via display information output unit 27. Biosignal analyzer 23b may also extract a signal having a specific frequency from the waveform-adjusted biosignal to calculate the power spectral density of the signal at such frequency.

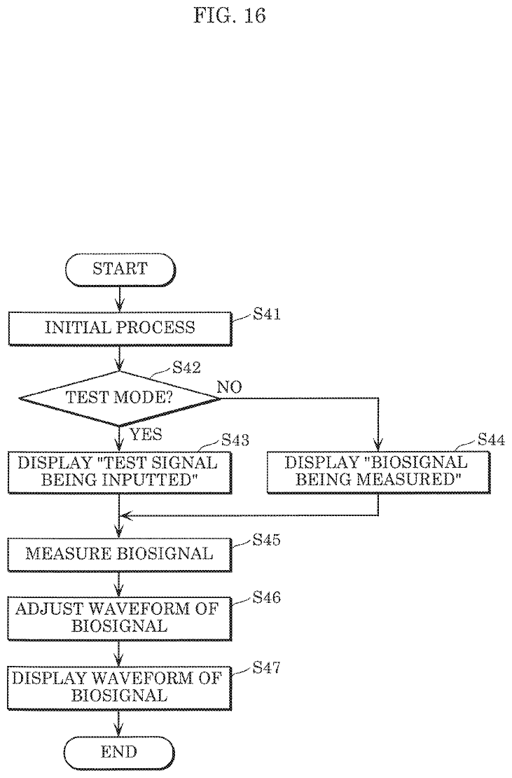

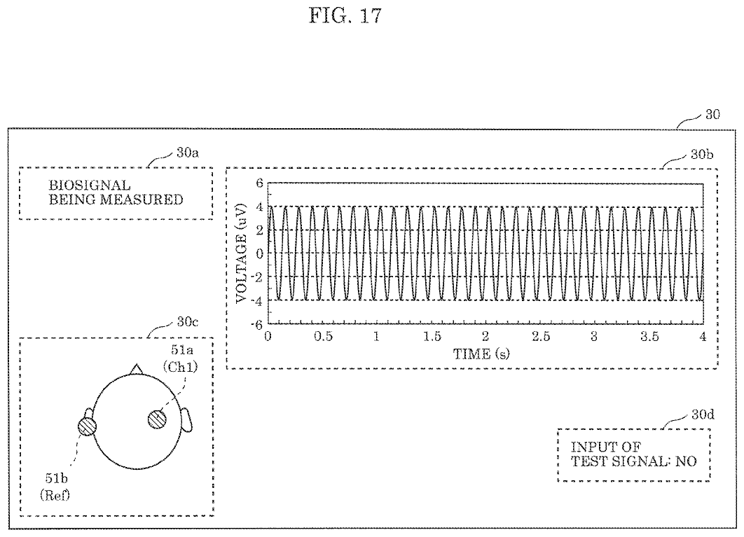

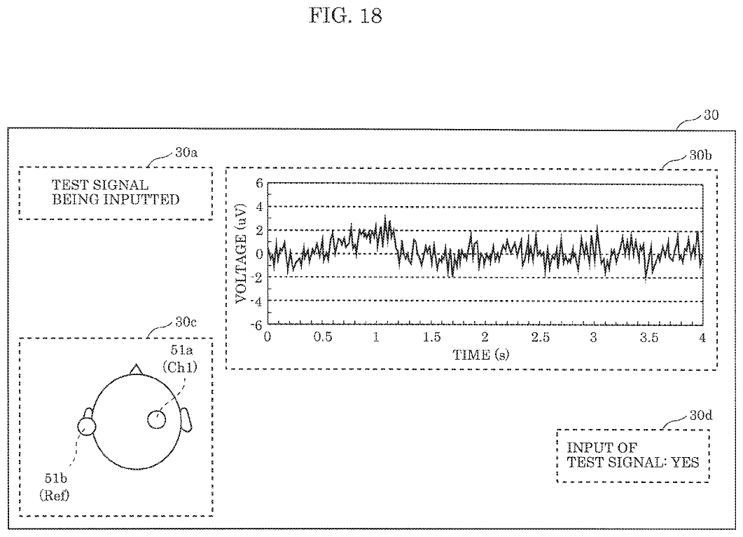

The following describes an exemplary display operation performed in biosignal measurement system 100. FIG. 16 is a flowchart of an exemplary display operation performed in biosignal measurement system 100. FIG. 17 is a diagram showing an exemplary display on presentation unit 30 in the biosignal measurement mode. FIG. 18 is a diagram showing an exemplary display on presentation unit 30 in the test mode.

First, app processing unit 26 performs an initial process (S41). As the initial process, as illustrated in FIG. 17 and FIG. 18, app processing unit 26 displays, on electrode illustration portion 30c of presentation unit 30, the positions and the connection states of measurement electrode 51a and reference electrode 51b included in headset 10 attached to test subject 5. The connection states indicate whether measurement electrode 51a and first chopper amplifier circuit 52a are connected, and whether reference electrode 51b and second chopper amplifier circuit 52b are connected. Stated differently, the connection sates are the ON/OFF states of switch element Sa1 and switch element Sb1. FIG. 17 and FIG. 18 represent the electrodes as hatched circles when the electrodes and the chopper amplifier circuits are connected, and represent the electrodes as circles without hatching when the electrodes and the chopper amplifier circuits are not connected.

Subsequently, app processing unit 26 determines whether the current operation mode is the test mode (S42). When an operation that designates the test mode is performed on operation button 41, as described above, app processing unit 26 determines whether operation signal obtainer 21 has obtained a notification signal that is transmitted from headset 10 in response to the operation that designates the test mode. When an operation that designates the test mode is performed on the user interface (not illustrated) included in information processing device 20, app processing unit 26 determines whether such operation has been performed.

When determining that the current operation mode is the test mode (YES in S42), app processing unit 26 displays “test signal being inputted” on measurement information display portion 30a in presentation unit 30 as illustrated in FIG. 18 (S43). App processing unit 26 also displays “input of test signal: YES” on signal input status display portion 30d in presentation unit 30.

Meanwhile, when determining that the current operation mode is not the test mode but the biosignal measurement mode (NO in S42), app processing unit 26 displays “biosignal being measured” on measurement information display portion 30a in presentation unit 30 as illustrated in FIG. 17 (S44). App processing unit 26 also displays “input of test signal: NO” on signal input status display portion 30d in presentation unit 30.

Subsequently biosignal measurement device 10b measures biosignals (here, brainwave signals) (S45), and each of the obtained biosignals is then transmitted to biosignal processing unit 23 via biosignal obtainer 22.

In biosignal processing unit 23 that has obtained the biosignal, biosignal waveform adjuster 23a adjusts the waveform of the biosignal (S46), and biosignal analyzer 23b performs signal processing such as filtering on the waveform-adjusted biosignal. Consequently, biosignal analyzer 23b outputs the biosignal waveform to app processing unit 26.

App processing unit 26 that has received the biosignal waveform displays the received biosignal waveform on biosignal waveform display portion 30b in presentation unit 30 as illustrated in FIG. 17 and FIG. 18 (S47).

As described above, in biosignal measurement system 100, the following items of information are displayed on presentation unit 30 in real time: measurement information display portion 30a representing the measurement information; biosignal waveform display portion 30b representing the biosignal waveform; electrode illustration portion 30c representing the positions of the electrodes; and test signal input status display portion 30d representing the input status of the test signal. This enables many items of information to be identified at a glance.

### [Active Electrode]

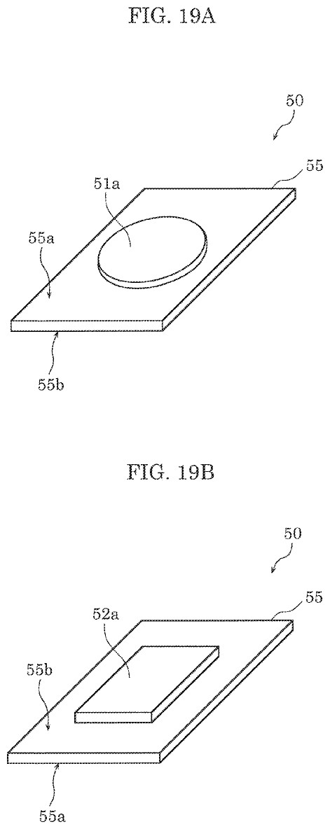

As illustrated in FIG. 19A and FIG. 19B, measurement electrode 51a and first chopper amplifier circuit 52a may be mounted on a single printed circuit board (PCB) 55 to be implemented as active electrode 50. FIG. 19A and FIG. 19B are schematic diagrams showing external views of active electrode 50. Measurement electrode 51a is mounted on one main surface 55a of PCB 55 as illustrated in FIG. 19A, and first chopper amplifier circuit 52a is mounted on the other main surface 55b of PCB 55 as illustrated in FIG. 19B. The structure in which measurement electrode 51a and first chopper amplifier circuit 52a are mounted on a single PCB 55 reduces the length of a wire that electrically connects measurement electrode 51a and first chopper amplifier circuit 52a. This eventually reduces the generation of unnecessary noise, etc.

Although not illustrated, reference electrode 51b and second chopper amplifier circuit 52b may also be mounted on a single PCB to be implemented as an active electrode.

### [Effect, Etc.]

As described above, biosignal measurement device 10b includes: first chopper amplifier circuit 52a that receives a biosignal detected by measurement electrode 51a contacting a living body, and is chopper-controlled based on first control signal CS1; and controller 60 that selectively performs one of a biosignal measurement mode operation for outputting first control signal CS1 having a first frequency to first chopper amplifier circuit 52a and a test mode operation for outputting first control signal CS1 having a different frequency from the first frequency to first chopper amplifier circuit 52a. Measurement electrode 51a is an example of the first electrode. The first frequency, an example of which is 2 kHz, may be defined empirically or experimentally such that first chopper amplifier circuit 52a generates a smaller amount of noise in the biosignal measurement mode.

Such biosignal measurement device 10b changes the frequencies of first control signal CS1, thereby outputting, as a test signal, the noise generated in first chopper amplifier circuit 52a. Biosignal measurement device 10b thus achieves an operation test that uses a test signal, while preventing an increase in the circuit scale.

Also, for example, controller 60 outputs first control signal CS1 having the first frequency of 0 Hz to first chopper amplifier circuit 52a in the test mode operation.

Such biosignal measurement device 10b stops chopper control, thereby outputting, as a test signal, the noise generated in first chopper amplifier circuit 52a.

Also, for example, biosignal measurement device 10b further includes second chopper amplifier circuit 52b that receives a biosignal detected by reference electrode 51b contacting the living body, and is chopper-controlled based on second control signal CS2. Controller 60 outputs second control signal CS2 having a second frequency to second chopper amplifier circuit 52b in the biosignal measurement mode operation, and outputs second control signal CS2 having a different frequency from the second frequency to second chopper amplifier circuit 52b in the test mode operation. Reference electrode 51b is an example of the second electrode. The second frequency, an example of which is 2 kHz, may be defined empirically or experimentally such that second chopper amplifier circuit 52b generates a smaller amount of noise in the biosignal measurement mode.

Such biosignal measurement device 10b changes the frequencies of second control signal CS2, thereby outputting, as a test signal, the noise generated in second chopper amplifier circuit 52b.

Also, for example, controller 60 outputs second control signal CS2 having the second frequency of 0 Hz to second chopper amplifier circuit 52b in the test mode operation.

Such biosignal measurement device 10b stops chopper control, thereby outputting, as a test signal, the noise generated in second chopper amplifier circuit 52b.

Also, for example, biosignal measurement device 10b further includes third chopper amplifier circuit 74 that amplifies a signal outputted from first chopper amplifier circuit 52a, and is chopper-controlled based on third control signal CS3. Controller 60 outputs third control signal CS3 having a third frequency to third chopper amplifier circuit 74 in the biosignal measurement mode operation, and outputs third control signal CS3 having a different frequency from the third frequency to third chopper amplifier circuit 74 in the test mode operation. The third frequency an example of which is 2 kHz, may be defined empirically or experimentally such that third chopper amplifier circuit 74 generates a smaller amount of noise in the biosignal measurement mode.

Such biosignal measurement device 10b changes the frequencies of third control signal CS3, thereby outputting the noise generated in third chopper amplifier circuit 74 as a test signal having a relatively great amplitude.

Also, for example, controller 60 outputs third control signal CS3 having the third frequency of 0 Hz to third chopper amplifier circuit 74 in the test mode operation.

Such biosignal measurement device 10b stops chopper control, thereby outputting, as a test signal, the noise generated in third chopper amplifier circuit 74.

Also, for example, controller 60 selectively performs one of the biosignal measurement mode operation and the test mode operation based on a signal that is obtained in response to a user operation. The signal that is obtained in response to the user operation is, for example, the signal indicating the test mode described in the above embodiment.

Such biosignal measurement device 10b is capable of switching between the biosignal measurement mode and the test mode in accordance with a user operation.

Also, for example, biosignal measurement device 10b further includes measurement electrode 51a, and PCB 55 on which measurement electrode 51a and first chopper amplifier circuit 52a are mounted.

Such biosignal measurement device 10b prevents the occurrence of unnecessary noise by reducing the length of the wire that electrically connects measurement electrode 51a and first chopper amplifier circuit 52a.

Also, the electroencephalograph, such as headset 10, includes: biosignal measurement device 10b; and attachment portion 40 which is attached to the head portion of the living body, and on which measurement electrode 51a is provided.

Such electroencephalograph changes the frequencies of first control signal CS1, thereby outputting, as a test signal, the noise generated in first chopper amplifier circuit 52a. The electroencephalograph thus achieves an operation test that uses a test signal, while preventing an increase in the circuit scale.

Furthermore, the control method of controlling biosignal measurement device 10b selectively performs one of the biosignal measurement mode operation for outputting first control signal CS1 having a first frequency to first chopper amplifier circuit 52a; and the test mode operation for outputting first control signal CS1 having a different frequency from the first frequency to first chopper amplifier circuit 52a.

Such control method changes the frequencies of first control signal CS1, thereby outputting, as a test signal, the noise generated in first chopper amplifier circuit 52a. The control method thus achieves an operation test that uses a test signal, while preventing an increase in the circuit scale.

### Another Embodiment

The embodiment has been described above, but the present invention is not limited to such embodiment.

For example, the circuit structure described in the embodiment is a mere example, and thus the present invention is not limited to the above circuit structure. Stated differently as in the case with the above circuit structure, the present invention also includes a circuit that achieves the characteristic functions of the present invention. For example, within a range in which the same functions are achievable as those achieved by the above circuit structure, the present invention also includes an element to which a switch element (transistor), a resistance element, a capacitative element, or another element is connected in series or in parallel.

In the above embodiment, a process performed by a specific processing unit may be performed by another processing unit. Also, the orders of a plurality of processes may be changed or a plurality of processes may be performed in parallel.

Also in the above embodiment, structural components such as the controller may be implemented by executing a software program suitable for each structural component. Each structural component may be implemented by a program executor such as a CPU and a processor reading out and executing the software program recorded in a hard disk, a semiconductor memory, or another recording medium.

The structural components such as the controller may be implemented as hardware. For example, the structural component such as the controller may be circuits (or integrated circuits). These circuits may be collectively implemented as a single circuit or individual circuits. These circuits may also be general-purpose circuits or dedicated circuits.

Also, general or specific aspects according to the present invention may be implemented as a system, a device, a method, an integrated circuit, a computer program, or a computer-readable recording medium such as a CD-ROM, or may be implemented as any combination of a system, a device a method, an integrated circuit, a computer program, and a recording medium.

For example, the present invention may be implemented as a control method of controlling the biosignal measurement device, or as a program that causes a computer to execute such program. The present invention may also be implemented as a non-transitory, computer-readable recording medium in which such program is recorded.

The biosignal measurement system described in the above embodiment may be implemented as a single device or as a plurality of devices. When the biosignal measurement system is implemented as a plurality of devices, the structural components included in the biosignal measurement system described in the above embodiment may be allocated to a plurality of devices in any manner.

Moreover, the scope of one or more aspects may include a variation achieved by making various modifications and alternations to the embodiment that can be conceived by those skilled in the art without departing from the essence of the present invention, and an aspect achieved by combining structural components in another embodiment.

### REFERENCE MARKS IN THE DRAWINGS

10 headset (electroencephalograph)
10b biosignal measurement device
40 attachment portion
51a measurement electrode (first electrode)
51b reference electrode (second electrode)
52a first chopper amplifier circuit
52b second chopper amplifier circuit
55 printed circuit board (PCB)
60 controller
74 third chopper amplifier circuit
CS1 first control signal
CS2 second control signal
CS3 third control signal

1. A biosignal measurement device, comprising:
a first chopper amplifier circuit that receives a biosignal detected by a first electrode contacting a living body, and is chopper-controlled based on a first control signal; and
a controller that selectively performs one of: a biosignal measurement mode operation for outputting the first control signal having a first frequency to the first chopper amplifier circuit; and a test mode operation for outputting the first control signal having a different frequency from the first frequency to the first chopper amplifier circuit.

2. The biosignal measurement device according to claim 1,
wherein the controller outputs the first control signal having the first frequency of 0 Hz to the first chopper amplifier circuit in the test mode operation.

3. The biosignal measurement device according to claim 1, further comprising:
a second chopper amplifier circuit that receives a biosignal detected by a second electrode contacting the living body, and is chopper-controlled based on a second control signal,
wherein the controller:
outputs the second control signal having a second frequency to the second chopper amplifier circuit in the biosignal measurement mode operation; and
outputs the second control signal having a different frequency from the second frequency to the second chopper amplifier circuit in the test mode operation.

4. The biosignal measurement device according to claim 3,
wherein the controller outputs the second control signal having the second frequency of 0 Hz to the second chopper amplifier circuit in the test mode operation.

5. The biosignal measurement device according to claim 1, further comprising:
a third chopper amplifier circuit that amplifies a signal outputted from the first chopper amplifier circuit, and is chopper-controlled based on a third control signal,
wherein the controller:
outputs the third control signal having a third frequency to the third chopper amplifier circuit in the biosignal measurement mode operation; and
outputs the third control signal having a different frequency from the third frequency to the third chopper amplifier circuit in the test mode operation.

6. The biosignal measurement device according to claim 5,
wherein the controller outputs the third control signal having the third frequency of 0 Hz to the third chopper amplifier circuit in the test mode operation.

7. The biosignal measurement device according to claim 1,
wherein the controller selectively performs one of the biosignal measurement mode operation and the test mode operation based on a signal that is obtained in response to a user operation.

8. The biosignal measurement device according to claim 1, further comprising:
the first electrode; and
a printed circuit board (PCB) on which the first electrode and the first chopper amplifier circuit are mounted.

9. An electroencephalograph, comprising:
the biosignal measurement device according to claim 1; and
an attachment portion which is attached to a head portion of the living body, and on which the first electrode is provided.

10. A control method of controlling a biosignal measurement device,
wherein the biosignal measurement device includes a first chopper amplifier circuit that receives a biosignal detected by a first electrode contacting a living body, and is chopper-controlled based on a first control signal, and
the control method comprises selectively performing one of: a biosignal measurement mode operation for outputting the first control signal having a first frequency to the first chopper amplifier circuit; and a test mode operation for outputting the first control signal having a different frequency from the first frequency to the first chopper amplifier circuit.

11. A non-transitory computer-readable recording medium for use in a computer, the recording medium having a computer program recorded thereon for causing the computer to execute the control method according to claim 10.
